SEQUENCE LISTING (1 sequence)

SEQ ID NO: 1 - DNA, 28 bp, Homo sapiens
aatttatttgtttttgccccttcccctt

(19) United States
(12) Patent Application Publication
(10) Pub. No.: US 2021/0332371 A1
(43) Pub. Date: Oct. 28, 2021

(54) NUCLEIC ACID TO ACTIVATE GENE EXPRESSION AND PROTEIN PRODUCTION

(71) Applicant: IBMC - INSTITUTO DE BIOLOGIA MOLECULAR E CELULAR, Porto (PT)

(72) Inventors: Marta OLIVEIRA, Porto (PT); Ana JESUS, Porto (PT); Jaime FREITAS, Porto (PT); Alexandra MOREIRA, Porto (PT); José Carlos RIBEIRO BESSA, Porto (PT)

(21) Appl. No.: 17/284,040

(22) PCT Filed: Oct. 9, 2019
(86) PCT No.: PCT/PT2019/050035

§ 371 Date: Apr. 9, 2021

(30) Foreign Application Priority Data
Oct. 9, 2018 (PT) 115073

(51) Int. Cl. C12N 15/67; A61K 48/00

(52) U.S. Cl. CPC C12N 15/67; A61K 48/0066

(57) ABSTRACT

A method for increasing the efficiency of protein production is described, said method employing a a nucleic acid with a specific non-coding nucleotide sequence, which strongly increases protein production in several eukaryotic systems, as well as the compositions that contain this nucleic acid sequence.

The nucleic acid sequence and method can be advantageously used as a new tool to boost protein production in biotechnology and/or biopharmaceutical applications, due to its ability to enhance protein production derived from a genetic construct, a gene expression or a reporter vector, in zebrafish, fruit fly or other model organisms, as well as in mammalian or human cells.

### TECHNICAL FIELD OF THE INVENTION

The present invention refers to a method for increasing the efficiency of recombinant protein production by employing a composition that comprises a nucleic acid with a specific nucleotide sequence which is used to incorporate in the sequence of a genetic construct or a gene expression vector, in zebrafish, fruit fly or other model organisms, as well as mammalian or human cells, to enhance protein production and be advantageously used as a new tool to boost protein yield in biotechnology and/or biopharmaceutical applications. Thus, the present invention falls within the technical field of chemistry, biochemistry, molecular biology, microorganism's genetic engineering, recombinant-DNA technology.

### STATE OF THE ART

The efficiency of recombinant protein production is an important bottleneck in most biopharmaceuticals and biotechnology industries and strategies to increase productivity usually rely on improvement in host cell growth media composition (as disclosed in documents US2017218328 and WO9429435) or on controlling the cell growth process, through induction or enhancement (as described in documents U.S. Pat. No. 5,089,397 and US2018135008, respectively); or through a stepwise process for scaling-up the culture. Another strategy to tackle this problem regards the use of more efficient expression vectors, trough addition of several types of expression enhancer elements, as described for example in documents CN107090441, CN107653266, KR20170140925 and CN104975018. The expression vectors are genetically engineered constructs for the introduction of genes and expression of proteins of interest in the host cells. Expression vectors may contain different types of promoters (as disclosed in US2019055580), as well as other regulatory elements, upstream and/or downstream of the gene coding sequence, so that more efficient expression of the gene of interest occurs. For example, document US2008241883 discloses enhancer elements for recombinant expression vectors to promote expression of a recombinant protein in a host cell. However, these elements are very long in length, with 2329 and 2422 base pairs, which makes them difficult to sub-clone and brings the drawback of adding considerable size to the backbone expression vector, making it more difficult to amplify, manipulate and transfect. Certain enhancers are functional only in specific host cells, as exemplified by the enhancer described in documents CN107653266 or CN105238816, which are designed only for insect cells, or the vector disclosed in US20110195451 which is designed only for animal cells. Other enhancer sequences are specific for given genes, as exemplified in documents US2010151520 and US2003013867. Thus, despite advances in the understanding of various sequences and other factors that can affect expression of recombinant proteins, needs remain to improve the yields of recombinant proteins, particularly of recombinant proteins that are intended for therapeutic or other specialized biotechnology applications. Desirably, this could be solved by small enhancer sequences that could be chemically synthesised and easily sob-cloned, with a “universal” effect in various types of host cells and different target genes without interfering with the gene coding region. A sequence with all these features, however, is still missing from the prior art.

These facts are disclosed in order to illustrate the technical problem addressed by the present disclosure.

### SUMMARY OF THE INVENTION

We found and characterized a new short 28 nucleotide sequence, herein named iPLUS—increase in Protein Levels Universal Sequence—and specified in SEQ ID NO: 1, that strongly increases protein production in vivo, in several model systems including fruit fly, zebrafish and in human cell lines. This sequence can be synthetized in vitro and used in compositions, vectors, recombinant cell lines or kits where it dramatically increases protein production, for example of the Polo protein or of the green fluorescent protein (GFP) protein. Through specific methods, the 28 nucleotide sequence, or smaller functional fragments of this sequence, can be inserted in the sequence of a gene, of an expression vector or of a reporter vector, to enhance protein production.

As such, the present invention refers to the use of a nucleic acid for increasing the expression of a protein of interest characterized by, comprising at least one copy of the 28-oligonucleotide with the base sequence of SEQ ID NO: 1.

In another embodiment of the present invention, the nucleic acid for increasing the expression of a protein of interest is characterized by, comprising at least one copy of a truncated sequence of the nucleotide base sequence SEQ ID NO: 1., between 4 and 27 nucleotides, most preferably 22 nucleotides.

Another aspect of the present invention refers to a composition for increasing the expression of a protein of interest characterized by, comprising at least one copy of a nucleic acid with the sequence SEQ ID NO: 1, or a truncated form of SEQ ID NO: 1, a buffer, such as Tris-HCl, a divalent ion chelating agent, such as EDTA, and combinations thereof.

In another embodiment of the present invention, the said compositions for increasing the expression of a protein of interest are characterized by, further comprising restriction enzyme recognition sequences upstream, downstream or at both ends of SEQ ID NO: 1 or of the truncated forms of SEQ ID NO: 1.

The present invention also refers to a genetic construct for increasing the expression of a protein of interest characterized by, comprising at least one copy of the nucleic acid with SEQ ID NO: 1, or a truncated form of SEQ ID NO: 1, downstream or upstream, most preferably downstream, of the gene of interest coding region.

In another embodiment of the present invention, the said genetic construct for increasing the expression of a protein of interest is an expression vector.

In another embodiment of the present invention, the said expression vector is selected from the group that consists of a recombinant eukaryotic plasmid expression vector, a recombinant prokaryotic expression vector, a recombinant viral expression vector, a recombinant bacteriophage expression vector, a recombinant yeast mini-chromosome expression vector, a recombinant bacterial artificial chromosome expression vector, and a recombinant yeast expression plasmid vector.

The present invention also refers to the host cell lines for increased expression of a protein of interest characterized by, comprising at least one exogenous copy of the nucleic acids described in SEQ ID NO: 1, introduced for example through gene editing.

In another embodiment of the present invention, the host cell lines for increased expression of a protein of interest are characterized by, comprising, stably or transiently, the said genetic constructs or expression vectors.

In another embodiment of the present invention, the host cell lines are selected from the group consisting of a mammalian host cell (non-limiting examples of which include a HeLa cell, a Chinese Hamster Ovary cell, a COS cell, a Vero cell, an SP2/0 cell, an NS/0 myeloma cell, a human embryonic kidney 293 cell, a baby hamster kidney cell, a human B cell, a human T cell Jurkat, a neuronal cell, a pancreatic cell, a CV-I /EBNA cell, an L cell, a 3T3 cell, an HEPG2 cell, and an MDCK cell), an insect host cell (non-limiting example of such includes Drosophila cells), a vertebrate host cell (non-limiting example includes zebrafish and zebrafish cell lines), a plant host cell (non-limiting example includes Nicotania tabacum, Arabidopsis, Lactuca sativa, protoplasts, suspension cells), a fungal host cell (non-limiting examples of which include Aspergillus, Neurospora, Saccharomyces, Pichia, Hansenula, Schizosaccharomyces, Kluyveromyces, Yarrowia, and Candida cells), an algal host cell (non-limiting example of which includes Synechocystis and other Cyanobacteria cells), a nematode host cell, a protozoan host cell, and a prokaryotic host cell (non-limiting examples of which include Escherichia coli, Erwinia chrysanthemi and Clostridium histolyticum cells).

Another aspect of the present invention refers to a method for increasing the expression of a protein of interest in a host cell characterized by comprising the steps of:
a) Synthesizing the nucleic acid insert with the oligonucleotide sequences described above and preparing the said compositions;
b) Contacting the insert nucleic acid composition with a linearized expression vector encoding for a protein of interest and ligating the insert through incubation with a DNA Ligase in the appropriate buffer and temperature conditions, so as to obtain an expression vector as mentioned above;
c) Introducing the said expression vectors or the said nucleic acid into a host cell, trough transient or stable transformation, transduction, transfection or trough gene editing protocols, so as to produce a host cell line; in accordance with claim 11.

In another embodiment of the present invention's method, the said insert is ligated downstream of the gene coding sequence, for transcription at the 3′ untranslated region (3′UTR).

In another embodiment of the present invention's method, the said insert is ligated upstream of the gene coding sequence, for transcription at the 5′ untranslated region (5′UTR).

In another embodiment of the present invention's method, the said insert is ligated both upstream and downstream of the gene coding sequence, for transcription both at the 5′UTR and at the 3′UTR.

In another embodiment of the present invention's method, the said protein of interest is selected from the group consisting of a kinase, such as the Polo kinase protein or other enzyme proteins, a reporter protein (such as GFP and Luciferase proteins), an antibody or an antibody fragment protein, an hormone protein, a growth factor protein, a cytokine protein, a receptor protein, a structural protein and a viral protein.

In another embodiment of the present invention's method, the said transformation, transduction, transfection or gene editing protocols include protocols known in the art that employ calcium chloride and heat-shock, viral particles, calcium phosphate, DEAE-dextran, electroporation, liposomes, non-liposomal lipids, dendrimers, guide RNAs and CRISPR/CAS9 enzymes or embryo microinjection.

The present invention also refers to a kit for increasing the expression of a protein of interest characterized by, comprising the said compositions prepared with the said nucleic acids, or the said genetic construct expression vectors, or the said host cell lines.

The present invention also refers to the use of at least one copy of a nucleic acid with the 28-oligonucleotide base sequence SEQ ID NO: 1. in a method for providing a therapy for a disease such as, but not limited to, cancer, genetic, pancreatic or neurologic disorders, through a gene editing method to introduce the said nucleic acid into host cells and increase the expression of a protein of interest.

Another aspect of the present invention refers to a method of lowering expression, substantially suppressing expression, or essentially shutting down expression of a protein of interest in a host cell comprising the step of: deleting or interfering with the non-coding nucleic acid with the 28-oligonucleotide base sequence of SEQ ID NO: 1.

The method, nucleic acid sequence, compositions, vectors, cell lines and kit of the present invention can be advantageously used as a new tool to boost protein production in biotechnology and/or biopharmaceutical applications, due to its ability to enhance protein production derived from a genetic construct, a gene expression or a reporter vector, in zebrafish, fruit fly or other model organisms, as well as in mammalian or human cells.

### BRIEF DESCRIPTION OF THE FIGURES

FIG. 1A: Embodiment in which iPLUS (SEQ ID NO: 1) increases GFP expression in zebrafish. Schematic representation of the construct used to generate the transgenic fish with the iPLUS inserted in the 3′UTR of GFP (pucl9 miniTOL-GFP-iPLUS)).

FIG. 1B: Embodiment in which iPLUS (SEQ ID NO: 1) increases GFP expression in zebrafish. The iPLUS activates GFP expression in comparison to the mutated iPLUS. Microinjection of pucl9 miniTOL-GFP-iPLUS and pucl9 miniTOL-GFP-iPLUSmt with mylz promotor mCherry in embryos at one cell stage. Images were acquired at 24 h stage.

FIG. 1C: Embodiment in which iPLUS (SEQ ID NO: 1) increases GFP expression in zebrafish. Quantification of iPLUS vs iPLUS mutant GFP expression. Analysis of GFP intensity of 30 embryos at 24 h stage, in each condition.

FIG. 1D: Embodiment in which iPLUS (SEQ ID NO: 1) increases GFP expression in zebrafish. Intense GFP expression in iPLUS founder Spot in comparison with the iPLUS mutant founder Zelya. Images were acquired at 48 h stage in dorsal and ventral view.

FIG. 2: Embodiment in which Luciferase activity is increased in HeLa cells transfected with iPLUS (SEQ ID NO: 1). Percentage of luciferase/Renilla (RLU) with different plasmids (pLucDelSV40, pLucDelSV40.iPLUS and pLucDelSV40.iPLUSmutant) in HeLa cells. The expression of Renilla and Luciferase is measured by the Dual Luciferase Reporter Assay (Promega) in a plate reader (Synergy 2).

FIG. 3A: Embodiment in which iPLUS (SEQ ID NO: 1) was deleted to decrease gene expression. Schematic representation of the transgene constructed to generate the transgenic flies wherein the iPLUS sequence is comprised at the 3′UTR, downstream of the polo gene. The arrow at the beginning of the transgene represents the endogenous polo promoter, the grey boxes represent the five polo exons, the black boxes represent the 5′ and 3′UTRs and the gfp box represents the gfp coding sequence inserted in frame with polo initiation codon. The hatched box in the 3′UTR represents the iPLUS sequence, which was deleted from the 3′UTR sequence.

FIG. 3B: Embodiment in which iPLUS (SEQ ID NO: 1) was deleted to decrease gene expression. GFP-Polo expression is decreased in flies with iPLUS deletion, as assessed by quantification of immunofluorescence of GFP-Polo in dividing neuroblasts; with Spc105 used as a kinetochore marker.

### DESCRIPTION OF THE INVENTION

We found and characterized a new short non-coding 28 nucleotide sequence, herein named iPLUS—increase in Protein Levels Universal Sequence—and specified in SEQ ID NO: 1, that strongly increases protein production in vitro and in vivo.

Because it is short, this sequence can be obtained by chemical synthesis and can be easily inserted into expression vectors or into the genome of host cells. Surprisingly, it demonstrates a “universal” protein expression enhancer effect, which is effective in various types of host cells and for different types of proteins of interest. Furthermore, because it is non-coding, its effect is achieved without interfering with the gene coding region and with the protein produced.

The said iPLUS sequence (SEQ ID NO: 1) can be prepared by nucleotide synthesis method using the solid phase phosphoramidite nucleosides as described in the publication: Beaucage, SL; Iyer, RP (1992). “Advances in the Synthesis of Oligonucleotides by the Phosphoramidite Approach”. Tetrahedron 48 (12): 2223. The synthesis can be performed on columns with solid support (controlled pore glass or polystyrene) functionalized with the first base of the 3′ end of each oligonucleotide. The preparation of the oligonucleotide follows after a number of synthesis cycles, each consisting of chemical reactions, typically four chemical reactions: i) release (detritylation), ii) coupling, iii) protection and iv) oxidation. In each cycle are added at the 5 ′ terminus of the growing chain, nucleotide residues corresponding to the desired sequence.

The nucleotides refer to nucleotides of natural or synthetic origin, with the hybridization capacity by base-pairing with complementary nucleotides, and may include, without limitation, DNA, RNA, and nucleotide analogues (e.g. nucleic acids with closed conformation, known as “locked nucleic acids”—LNA) nucleotides or without inter-nucleotide phosphodiester type linkages (e.g. peptide nucleic acid—PNA) and nucleic acids with tiodiester bonds, or the like for the same purpose.

In another embodiment of the present invention, smaller truncated functional fragments of this sequence (SEQ ID NO: 1), between 4 and 27 nucleotides, most preferably 22 nucleotides, can be inserted in the sequence of a gene, of an expression vector or of a reporter vector, to enhance protein production.

In another embodiment of the present invention, more than one copy of the nucleic acid of SEQ ID NO: 1 or of the smaller truncated functional fragments of this sequence can be used in tandem repeats, in order to achieve higher fold-changes in protein production.

In another embodiment of the present invention, the nucleic acid with SEQ ID NO: 1 or the smaller functional fragments of this sequence are used to generate a composition further comprising buffer, such as Tris-HCl, a divalent ion chelating agent, such as EDTA, or combinations thereof, for inserting the sequences into expression vectors or host cells.

The insertion of the sequence increasing protein production into expression or reporter vectors can be performed by DNA Ligase-mediated phosphodiester bonds between DNA fragments with “blunt ends”.

In an embodiment of the present invention, a vector, we termed pLuc with similar features to the vector pmirGLO (from Promega), which expresses the reporter enzyme Luciferase, is linearized through Smal digestion (1 hr at 37° C.) and dephosphorylated with 2 pl of FastAP during 10 minutes at 37° C. Insert iPLUS nucleotide sequence (SEQ ID NO: 1) or a mutant form (iPLUS mutant),used as control, and the linearized pLuc are ligated, using 100 ng of pLuc and 19 ng of iPLUS or iPLUS mutant (5:1), at 22° C. for 2 h, in the presence of 5 units of T4 DNA Ligase.

In an embodiment, GFP-iPLUS and GFP-iPLUS mutant sequences are subcloned into SalI and Kpnl restriction enzyme sites of the pUC19miniTOL expression vector by molecular biology methods employing compositions comprising the 28 nucleotide enhancing sequence.

Thus, in another embodiment of the present invention, the nucleic acid sequence (SEQ ID NO: 1) in the insert composition can further comprise recognition sequences restriction enzymes upstream, downstream or at both ends of SEQ ID NO: 1, to provide “sticky-ends” for more efficient and oriented insert ligation.

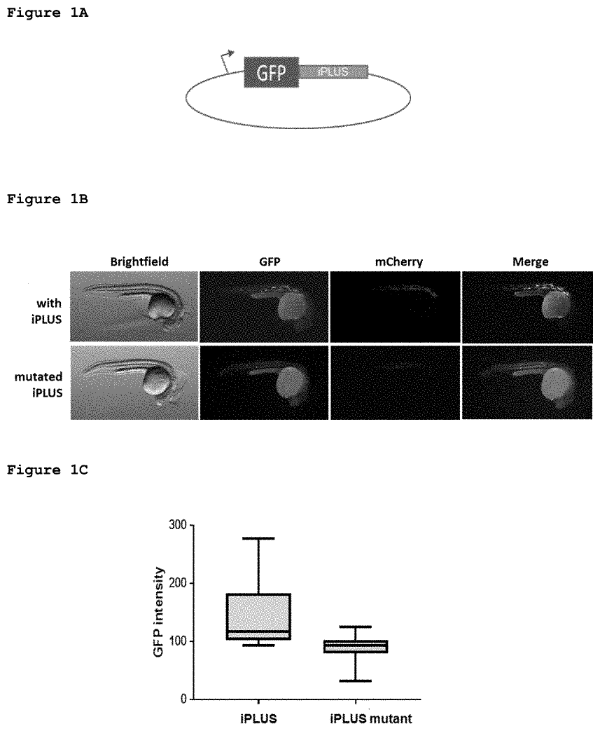

In another embodiment of the invention, the iPLUS is subcloned downstream of GFP and the resultant construct (pucl9 miniTOL-GFP-iPLUS, FIG. 1A) is microinjected into zebrafish one cell stage embryos. A control construct is also made where the iPLUS sequence is mutated (pucl9 miniTOL-GFP-iPLUSmt). Notably, GFP protein production is increased in zebrafish embryos microinjected with the iPLUS in comparison to the iPLUS mutant control, visualized by fluorescence microscopy (FIG. 1B). Quantification of protein level clearly shows that the iPLUS enhances GFP production in contrast to control (FIG. 1C). Zebrafish where GFP-iPLUS and GFP-iPLUS mutant were randomly inserted into the genome confirm that the iPLUS acts as an activator for protein production (FIG. 1D).

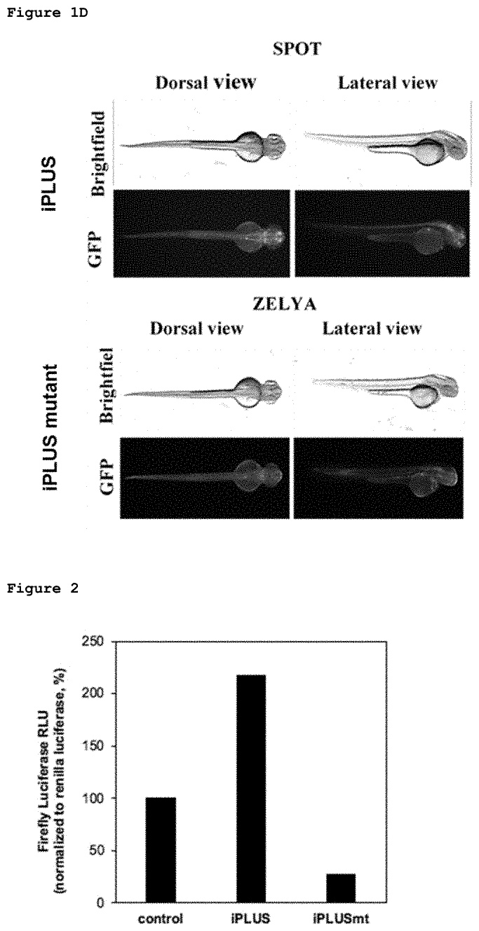

In another embodiment of the invention, the iPLUS sequence (SEQ ID NO: 1) is sub-cloned downstream of the luciferase gene and the resultant construct (pLucDelSV40.iPLUS) is transfected into HeLa cells. A control construct was also designed where the iPLUS was mutated (pLucDelSV40.iPLUSmutant). Levels of Luciferase protein activity were quantified and results show that there is an eight-fold increase in cells transfected with the iPLUS in comparison to control (FIG. 2).

Thus, in the preferred embodiment of the present invention comprises the expression vectors in which iPLUS (SEQ ID NO: 1) is inserted downstream of the encoded gene of interest (LUC or GFP in the former case).

The genetic constructs, such as expression vectors generated in the above mentioned manner constitute another embodiment of the present invention, which refers to the genetic constructs and expression vectors in which the SEQ ID NO: 1, tandem repeats, or smaller truncated functional fragments, are embodied.

Expression vectors in which iPLUS can be inserted may comprise recombinant a recombinant eukaryotic plasmid expression vector, a recombinant prokaryotic expression vector, a recombinant viral expression vector, a recombinant bacteriophage expression vector, a recombinant plant expression vector, a recombinant yeast mini-chromosome expression vector, a recombinant bacterial artificial chromosome expression vector, and a recombinant yeast expression plasmid vector.

In another embodiment, the above mentioned genetic constructs or vectors with at least one copy of a nucleic acid with the 28-oligonucleotide base sequence SEQ ID NO: 1. can also be used as reporters for testing the activity of the 28 nucleotide sequence, or smaller functional fragments of this sequence, to find modulators of its efficiency, including other genes, chemical compounds, or genetic modifications within host cells.

In another embodiment, competent bacteria are transformed with the ligation product comprising the plasmid expression vector embodying iPLUS (SEQ ID NO: 1) by heat shock at 42° C. for 1 min. Colonies harboring the iPLUS and iPLUS mutant are selected and plasmid DNA is obtained. Expression vector plasmids are then transfected into HeLa cells cultured in Dulbecco's modified Eagle's medium (DMEM 1× GlutaMAX, Gibco) supplemented with 10% (v/v) Fetal Bovine Serum (FBS) (Gibco), with the Lipofectamine 3000 Reagent (from Thermo Fisher Scientific) according to a manufacturer's protocol.

In another embodiment, a mixture is microinjected into one cell stage zebrafish embryos containing 50 ng of pUC19miniTOL-GFP-iPLUS and pUC19miniTOL-GFP-iPLUS mutant control, plus 50 ng of Tol2 and 0.1 μL of PhenolRed (used as a dye to guide microinjection success). After microinjection, the embryos are placed in a E3 medium (28.67 g of NaCl, 1.26 g of KCl, 4.83 g of CaCl2.2H2O, 8.17 g of MgSO4.7H2O, 1,5 mL of methylene blue 0.1% filling until 1 L of H2O). After two hours post fertilization (hpf) the embryos are transferred to a PTU medium (600 mg of N-phenylthiourea dissolved in 200 mL of H2O). After 24 hpf, the embryos are bleached out and incubated at 28° C. during the next first five days and then the embryos are transferred to small tanks with a low volume of water, being feed three times per day through a droplet mechanism.

Embryos are then put in a Petri dish with agarose and PTU medium, and the chorion is removed. Tricaine MS222 (from Sigma) at concentration 0.168 mg/ml is used to anesthetize the embryos according to good ethic practices. A stereomicroscope Leica M205 is used to screen embryos that expressed GFP and analyse the embryos microinjected with mCherry and pUC19-GFP-iPLUS or pUC19-GFP-iPLUS mutant control.

Thus, several types of host cells may be transfected with expression vectors comprising iPLUS for increased protein expression and, as such, other embodiments of the present invention refer to the host cell lines for increased expression of a protein of interest that comprise, stably or transiently, the expression vectors that integrate iPLUS (SEQ ID NO: 1).

Overall, the range of host cell lines generated by transfecting the iPLUS sequence (SEQ ID NO: 1) may comprise a mammalian host cell (non-limiting examples of which include a HeLa cell, but also a Chinese Hamster Ovary cell, a COS cell, a Vero cell, an SP2/0 cell, an NS/0 myeloma cell, a human embryonic kidney 293 cell, a baby hamster kidney cell, a human B cell, a human T cell Jurkat, a neuronal cell, a CV-I/EBNA cell, an L cell, a 3T3 cell, an HEPG2 cell, and an MDCK cell), an insect host cell (non-limiting example of such includes Drosophila cells), a fish host cell (non-limiting example includes zebrafish one cell embryo), a plant host cell (non-limiting example includes Nicotania tabacum, Arabidopsis, Lactuca sativa, protoplasts, suspension cells) a fungal host cell (non-limiting examples of which include Aspergillus, Neurospora, Saccharomyces, Pichia, Hansenula, Schizosaccharomyces, Kluyveromyces, Yarrowia, and Candida cells), an algal host cell (non-limiting example of which includes Synechocystis and other Cyanobacteria cells), a nematode host cell, a protozoan host cell, and a prokaryotic host cell (non-limiting examples of which include Escherichia coli, Erwinia chrysanthemi and Clostridium histolyticum cells).

Aside from host cell lines embodying an expression vector comprising iPLUS (SEQ ID NO: 1) another embodiment of the present invention refers to host cell line comprising at least one exogenous copy of the nucleic acid with sequence SEQ ID NO: 1, not necessarily integrated into an expression vector, which is introduced downstream of a gene of interest for example through gene editing protocols known in the art, and remains under the control of the native gene regulator elements.

According to the above mentioned embodiments, another aspect of the present invention refers to a method for increasing the expression of a protein of interest in a host cell characterized by comprising the steps of:
a) Synthesizing the nucleic acid insert with the oligonucleotide sequences described above and preparing the said compositions;
b) Contacting the insert nucleic acid composition with a linearized expression vector encoding for a protein of interest and ligating the insert through incubation with a DNA Ligase in the appropriate buffer and temperature conditions, so as to obtain an expression vector as mentioned above;
c) Introducing the said expression vectors or the said nucleic acid into a host cell, trough transient or stable transformation, transduction, transfection or trough gene editing protocols, so as to produce a host cell line;

Also, according to the herein described embodiments, the said protein of interest may be selected from the group consisting of a kinase, such as the Polo kinase protein or other enzyme proteins, a reporter protein (such as GFP and Luciferase proteins), as well as other common recombinant proteins such as an antibody or an antibody fragment protein, an hormone protein, a growth factor protein, a cytokine protein, a receptor protein, a structural protein and a viral protein.

According to the herein described embodiments, in the present invention's method, the said transformation, transduction, transfection or gene editing protocols include protocols known in the art that employ calcium chloride competent bacteria and heat-shock, liposomes such as the Lipofectamine 3000 reagent, direct, as well as other protocols known in the art comprising viral particles, calcium phosphate, DEAE-dextran, electroporation, non-liposomal lipids, dendrimers and guide RNAs and CRISPR/CAS9 enzymes.

According to the herein described embodiments, the present invention also refers to a kit for increasing the expression of a protein of interest comprising at least one of the embodiments for conducting the present invention's method, namely the above mentioned compositions prepared with the iPLUS nucleic acids with SEQ ID NO: 1, or the above mentioned expression vectors, or the host cell lines in which these vectors are embodied.

Due to the fact that iPLUS is a short sequence, it is amenable to integration into host cell lines, including embryo cells, through gene editing protocols known in the art, and thus the present invention also refers to the use of at least one copy of a nucleic acid with the 28-oligonucleotide base sequence SEQ ID NO: 1. in a method for providing therapy of a disease such as, but not limited to, cancer, genetic, pancreatic or neurologic disorders, through a gene editing method to introduce the exogenous nucleic acid into host cells and increase the expression of a protein of interest.

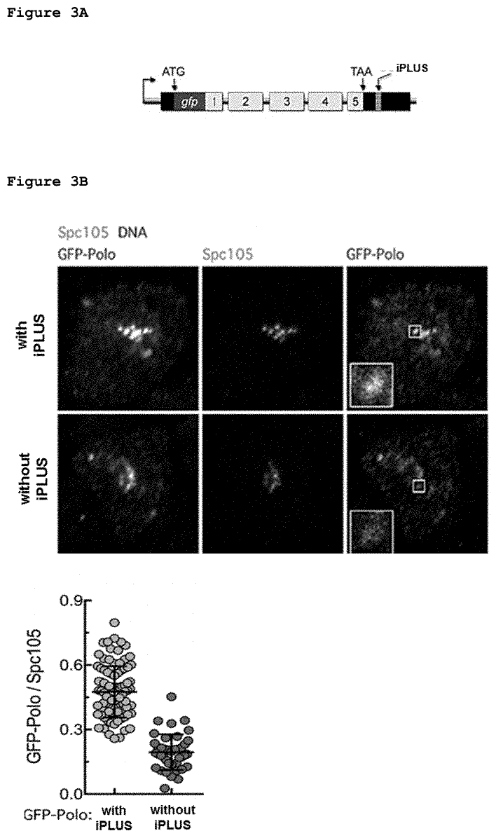

In one embodiment of the present invention, the polo kinase protein levels were quantified in transgenic flies by confocal microscopy. Transgenic flies carrying pW8-gfp-polo kinase protein with either iPLUS or a deleted iPLUS sequence (gfp-polo-Del-iPLUS are obtained by injection of ww1118 embryos with the respective transgenes (FIG. 3A). Selection of transgenic lines with the transgene on the second chromosome was performed by mating the transgenic flies with a strain carrying dominant markers and a balancer chromosome, w1118; Sco/SM6. These are then mated with w1118; If/CyO; MKRS/TM6B and w1118; If/CyO; polo9/TM6C to generate the w1118; gfp-polo-Del-iPLUS; polo9/TM6B line (herein referred as Del-iPLUS transgenic fly strain). Dissected larvae brains from each strain are placed in fresh 50 pM colchicine in PBS for 1 h30 at 25° C. and then fixated for 5 min with 1.8% formaldehyde and 45% acetic acid. Brain squashes are incubated with absolute ethanol for 10 min, then 10 min with PBS 0.1% Triton X-100 and a 10 min wash with PBS. Fixated brains are blocked with 10% FBS in PBS-T 0.05% for lh at RT and then incubated overnight at 4° C. with the following antibodies: rat anti-Spc105 (1:150), mouse anti-Polo (1:10, MA294), rabbit anti-P-Aurora (1:500, Rockland, Limerik, Pa.), rabbit anti-Thr676-P-Mpsl (1:10000) and guinea pig anti-total Mps1 (1:250, Gp15 RRID:AB 2567774). This is followed by three 5 minutes washes in PBS 0.05% Tween20 and incubation with the following secondary antibodies (Thermo Fischer) diluted 1:250 in PBS 0.05% Tween20 for 2 h at RT: goat anti-rat AlexaFluor 647, goat anti-mouse AlexaFluor 488, goat anti-rabbit AlexaFluor 568 and goat anti-guinea pig AlexaFluor 568. Image acquisition is performed using a Laser Scanning confocal microscope Leica TCS SP5 II and the LAS 2.6 software (Leica Microsystems, Germany). Results of Polo protein expression in vivo clearly show a strong reduction in protein production in transgenic flies without iPLUS (FIGS. 3B and 3C).

Thus, another aspect of the present invention refers to a method of lowering expression, substantially suppressing expression, or essentially shutting down expression of a protein of interest in a host cell comprising a step of deleting or interfering with the nucleic acid with the 28-oligonucleotide base sequence of SEQ ID NO: 1.

The nucleic acid sequence, compositions, vectors, host cell lines, methods, kit and uses of the present invention can be advantageously used as a new tool to boost protein production in biotechnology and/or biopharmaceutical applications, due to its ability to enhance protein production derived from a genetic construct, a gene expression or a reporter vector, in zebrafish, fruit fly or other model organisms, as well as in mammalian or human cells.

In conclusion, the iPLUS, a 28 nucleotide nucleic acid sequence, is a new activator of protein production in vivo and in vitro in different model organisms comprising fruit fly, zebrafish and in human cells. The iPLUS can be easily manipulated by use of compositions, methods and kits to be used as an effective tool to boost production of proteins with in biotechnology and/or biopharmaceutical applications.

All patents, applications, and publications cited in the above text are included herein by reference.

Other variations and embodiments of the invention described herein will now be apparent to those skilled in the art, and all such variants and alternative embodiments of the invention are intended to be encompassed within the foregoing description and the claims that follow.

SEQUENCE LISTING

SEQ ID NO: 1: 5′-AATTTATTTGTTTTTGCCCCTTCCCCTT-3′
Porto, Oct. 9^{th}, 2019

1. A nucleic consisting of at least one copy of the 28-oligonucleotide with the base sequence of SEQ ID NO: 1.

2. (canceled)

3. A composition for increasing the expression of a protein of interest comprising: (i) a nucleic acid consisting of at least one copy of a nucleic acid with the sequence SEQ ID NO: 1, as described in claim 1, or a nucleic acid consisting of at least one copy of the sequence SEQ ID NO: 1 having restriction enzyme recognition sequences upstream, downstream or at both ends of the at least one copy of SEQ ID NO: 1 described in claim 1; (ii) a buffer; (iii) a divalent ion chelating agent; and (iv) combinations thereof,

4. The composition according to claim 2, wherein the buffer is Tris-HCl, the divalent ion chelating agent is EDTA, or both.

5. A genetic construct for increasing the expression of a protein of interest, comprising at least one copy of the nucleic acid with SEQ ID NO: 1, as described in claim 1, downstream of the gene of interest coding region, wherein the genetic construct for increasing recombinant protein expression is an expression vector selected from the group consisting of a recombinant eukaryotic plasmid expression vector, a recombinant viral expression vector, a recombinant bacteriophage expression vector, a recombinant yeast mini-chromosome expression vector, a recombinant plant vector, a recombinant bacterial artificial chromosome expression vector, and a recombinant yeast expression plasmid vector.

6. (canceled)

7. (canceled)

8. A host cell for increased expression of a protein of interest, comprising at least one exogenous copy of the nucleic acid described in claim 1, introduced downstream of the gene of interest coding region through gene editing, wherein the host cell is selected from the group consisting of a mammalian host cell, a fish host cell, a plant host cell, a fungal host cell, an algal host cell, a nematode host cell, and a protozoan host cell.

9. Host cells for increased expression of a protein of interest comprising, stably or transiently, the genetic constructs or expression vectors described in claim 5.

10. (canceled)

11. A method for increasing the expression of a protein of interest in a host cell comprising the steps of:
a) synthesizing a nucleic acid for increasing the expression of a protein of interest comprising at least one copy of the 28-oligonucleotide with the base sequence of SEQ ID NO: 1 and preparing compositions comprising said nucleic acid, a buffer, a divalent ion chelating agent, and combinations thereof;
b) contacting the nucleic acid composition with a linearized expression vector encoding for a protein of interest and ligating the nucleic acid downstream of the gene coding sequence, for transcription at the 3′ untranslated region (3′UTR) through incubation with a DNA Ligase in the appropriate buffer and temperature conditions, so as to obtain an expression vector comprising at least one copy of the nucleic acid with SEQ ID NO: 1 downstream of the gene of interest coding region;
c) introducing the said expression vectors or the nucleic acid for increasing the expression of a protein or interest comprising at least one copy of the 28-oligonucleotide with the base sequence of SEQ ID NO: 1 into a host cell, trough transient or stable transformation, transduction, transfection or through gene editing protocols, wherein said nucleic acid is introduced into the host cell downstream of the gene of interest coding region, so as to produce a host cell such as the host cell comprising at least one exogenous copy of the nucleic acid for increasing the expression of a protein of interest comprising at least one copy of the 28-oligonucleotide with the base sequence of SEQ ID NO: 1, introduced through gene editing; and
d) expressing the protein of interest.

12. (canceled)

13. (canceled)

14. (canceled)

15. The method according to claim 11 wherein the said protein of interest is selected from the group consisting of a kinase or other enzyme proteins, a reporter protein, an antibody or an antibody fragment protein, a hormone protein, a growth factor protein, a cytokine protein, a receptor protein, a structural protein and a viral protein.

16. The method according to claim 11 wherein the said transformation, transduction, transfection or gene editing protocols employ calcium chloride and heat-shock, viral particles, calcium phosphate, DEAE-dextran, electroporation, liposomes, non-liposomal lipids, dendrimers, guide RNAs and CRISPR/CAS9 enzymes or embryo microinjection.

17. A kit for increasing the expression of a protein of interest, comprising the composition of claim 3.

18. A method for treating a disease selected from cancer, genetic and neurologic disorders, through a gene editing method to introduce a nucleic acid comprising the 28-oligonucleotide base sequence SEQ ID NO: 1 into host cells and increase the expression of a protein of interest, wherein the nucleic acid is introduced downstream of the gene of interest coding region.

19. A method of lowering expression, substantially suppressing expression, or essentially shutting down expression of a protein of interest in a host cell comprising the step of: deleting or interfering with the nucleic acid with the 28-oligonucleotide base sequence of SEQ ID NO: 1.

20. The host cell of claim 8, wherein the mammalian host cell is selected from the group consisting of a HeLa cell, a Chinese Hamster Ovary cell, a COS cell, a Vero cell, an SP2/0 cell, an NS/0 myeloma cell, a human embryonic kidney 293 cell, a baby hamster kidney cell, a human B cell, a human T Cell Jurkat, a neuronal cell, a CV-I /EBNA cell, an L cell, a 3T3 cell, an HEPG2 cell, and an MDCK cell; wherein the fish host cell includes zebrafish; wherein the plant host cell is selected from the group consisting of Nicotania tabacum, Arabidopsis, Lactuca sativa, protoplasts, and suspension cells; wherein the fungal host cell is selected from the group consisting of Aspergillus, Neurospora, Saccharomyces, Pichia, Hansenula, Schizosaccharomyces, Kluyveromyces, Yarrowia, and Candida cells; and wherein the algal host cell is selected from the group consisting of Synechocystis and Cyanobacteria cells.

21. The method according to claim 8, wherein the protein of interest is selected from the group consisting of a Polo kinase protein, a GFP protein and a luciferase protein.
